(19) United States
(12) Patent Application Publication
(10) Pub. No.: US 2004/0068762 A1
(43) Pub. Date: Apr. 8, 2004

(54) Transgenic non-human mammals expressing a reporter nucleic acid under the regulation of androgen response elements

(76) Inventors: Ricardo M. Attar, Lawrenceville, NJ (US); David K. Bol, Gaithersburg, MD (US); Marco Gottardis, Princeton, NJ (US); Kasim Mookhtiar, Richboro, PA (US); Ronald Bruce Rowley, Guilford, CT (US); Jacek Ostrowski, Jamison, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/620,514

(22) Filed: Jul. 16, 2003

Related U.S. Application Data
(60) Provisional application No. 60/396,501, filed on Jul. 17, 2002.

(51) Int. Cl. A01K 67/027; C12N 5/06

(52) U.S. Cl. 800/18; 435/354

(57) ABSTRACT

A transgenic non-human mammal whose genome comprises a nucleic acid construct, wherein said construct comprises a reporter nucleic acid encoding a reporter operably linked to a promoter comprising an androgen response element (ARE), and said construct further comprises an androgen receptor nucleic acid encoding an androgen receptor, and wherein expression of said reporter nucleic acid is regulated by expression of said androgen receptor nucleic acid. The transgenic non-human mammals can be used as an in vivo model for the identification and development of selective androgen receptor modulators (SARMs) for the treatment of cancer or other disorders associated with defective androgen receptor function.

This application claims the benefit of U.S. Provisional Application No. 60/396,501 filed July 17, 2002, whose contents are incorporated by reference in its entirety.

### FIELD OF THE INVENTION

The present invention is directed towards a transgenic non-human mammal for the in vivo evaluation of androgen receptor function. The invention further pertains to the use of such mammals in the development of compounds and therapies that modulate androgen receptor activity.

### BACKGROUND OF THE INVENTION:

Recent advances in recombinant DNA and genetic techniques have made it possible to introduce and express a desired gene sequence in a recipient animal. Through the use of such methods, animals have been engineered to carry non-naturally occurring sequences or genes, that is, sequences or genes that are not normally or naturally present in the unaltered animal. The techniques have also been used to produce animals which exhibit altered expression of naturally present gene sequences.

Animals produced through the use of these methods can be either “chimeric” in which only some of the animal's cells contain and express the introduced sequence or gene or “transgenic” in which all of the cells of the animal contain the introduced sequence or gene. Consequently, in the case of transgenic animals every animal is capable of transmitting the introduced genetic material to its progeny as compared to the chimeric animals in which transmittal to progeny is dependent upon whether the introduced material is present in the germ cells of the animal.

The high efficiency transformation of cultured mammalian cells has been accomplished by direct microinjection of specific DNA sequences into the cell nucleus (M. Capecchi, Cell 22:479-488 (1980)). More specifically, it has also been demonstrated that DNA could be microinjected into mouse embryos and found in the resultant offspring (J. Gordon et al., P.N.A.S. U.S.A. 77:7380-7384 (1980)). Thus, the ability to produce certain transgenic mice is described and well known in the art.

The basic procedure for producing transgenic mice requires the recovery of fertilized eggs from newly mated female mice and then microinjecting into the male pronucleus of said egg the DNA that contains the sequence or gene to be transferred into the mouse. The microinjected eggs are then implanted in the oviducts of one-day pseudopregnant foster mothers and allowed to proceed to term. The newborn mice are then tested for the presence of the microinjected DNA by means known in the art and appropriate to detect the presence of the microinjected DNA. See, for example, T. Wagner et al., P.N.A.S. U.S.A. 78:6376-6380 (1981), U.S. Pat. No. 4,873,191, which describes the production of mice capable of expressing rabbit beta-globin in its erythrocytes.

Androgens are steroid hormones mainly responsible for male sexual characteristics during development and in adulthood. In a normal adult man, approximately five to seven milligrams per day of testosterone (T), the principal androgen, are produced and released by the testis into the systemic circulation. testosterone or its more potent metabolite, dihydrotestosterone (DHT) (K. Sundaram, Steroid Biochem. Mol. Biol. 53:253-257 (1995)), binds to the androgen receptor (AR), a member of the steroid nuclear-hormone-receptor (NHR) superfamily. These intracellular receptors are ligand-dependent transcription factors that regulate the transcription of a variety of genes. The AR is widely distributed among reproductive and non-reproductive tissues, including the prostate and seminal vesicles, male and female genitalia, skin, testis, ovary, cartilage, sebaceous glands, hair follicles, sweat glands, cardiac muscle, skeletal and smooth muscle, gastrointestinal vesicular cells, thyroid follicular cells, adrenal cortex, liver, pineal, and numerous brain cortical and subcortical regions, including spinal motor neurons (A. Negro-Vilar, J. Clin. Endocrinol. Metab., 54(10):3459-62 (1999)). Testosterone can also be metabolized in secretory and target organs to oestrogen (oestradiol-17β) by aromatases (M. Sawaya et al., J. Invest. Dermatol. 109: 296-300 (1997), C. Roselli et al., Biol. Reprod. 58: 79-87 (1998)), and affect gene expression through the estrogen receptor.

The effects of androgens are multiple. In the embryo, they are responsible for the differentiation of the reproductive organs into the male phenotype. During puberty, the increase in androgen production induces the secondary sexual characteristics. In addition, androgens also affect other aspects of human life such as social behavior, sexuality and physical appearance.

For example, it has been reported that castration, which causes a cessation of testosterone, leads to a decrease in the aggression in animals (J. Morley, Handbook of Clinical Psychoneuroendocrinology, Nemroff, C. B. and Loosen, P. T. (editors), Guilford, N.Y., pp. 3-41 (1987)). Low testosterone levels have also been associated with fatigue, while testosterone replacement often produces a general feeling of well being Nolten W. E., Curr Urol Rep. 4:313-9 (2000). It has been shown that testosterone improves memory in male mice (J. Flood, P.N.A.S. U.S.A. 89:1567-1571 (1992)), and two placebo controlled studies in older males have found that testosterone improves visuospatial cognition (J. Jankowsky, Behav. Neurosci. 108:325-332 (1994)). In addition, it is believed that androgens have an effect on the vascular system in that studies suggest an inverse relationship between the free testosterone plasma levels in men and the degree of coronary heart disease in these subjects (G. Phillips et al., Arterioscler. Thromb. 14:701-706 (1994), K. English, Eur. Heart J. 21:890-894 (2000)). Infusion of testosterone into the coronary arteries of men with coronary artery disease results in an acute significant increase in coronary blood flow (C. Webb et al., Circulation 100:1690-1696 (1999)). Androgens have also been shown to have beneficial effects on endothelial cell function (Ong et al., Ann. J. Cardiol. 85:14-17 (2000)) and myocardial ischaemia (C. Webb et al., Ann. J. Cardio. 83:437-439 (1999); K. English, Circulation 102:1906-1911 (2000)). In terms of sexuality, testosterone clearly improves libido both in cross-sectional (R. Schiavi et al., Psychosom. Med. 53:363-374 (1991)) and in interventional studies (Morales et al., J. Urol. 157(3):849-854 (1997); I. Klepsch et al., Endocrinologie 20(4):289-293 (1982)). Regarding the effect of androgens on appearance and body composition, there are several studies in men that support the concept that testosterone improves muscle strength in older males with low levels of testosterone (R. Baumgartner et al., Mech. Ageing Dev. 107(2):123-136 (1999); R. Sih et al., J. Clin. Endocr. Metab. 82:1661-167 (1997); R. Orrell et al., J. R. Soc. Med. 88:454-46 (1995)). In terms of the effects of androgens on bone, it has been shown that bone mineral density declines with age in men (H. Burger et al., Ann. J. Epidem. 147(9):871-879 (1998)), and is increased in hypogonadal older males receiving testosterone replacement therapy (P. Snyder et al., J. Clin. Endocr. Metab. 84:1966-1972 (1999); I. Reid et al., Arch. Intern. Med. 156:1173-117 (1996)).

Despite the lack of a precise understanding of the mechanisms by which androgens act on so many physiological relevant systems, it is readily understood why the AR is an important target in multiple areas of drug discovery and patient therapy. In the oncology area, for example, inhibitors (antagonists or partial antagonists) of androgen receptor function are useful for the treatment of androgen dependent prostate cancer while agonists or partial agonists of the AR are applicable to the treatment of breast cancer. For metabolic and endocrine diseases disorders, agonists or partial agonists of the androgen receptor function are useful for the treatment of age-related diseases and conditions of cachexia in several disease states including, but not limited to, Acquired Immune Disease Syndrome (AIDS). Functional AR has also been identified in various bone cells and, as such, androgen administration has beneficial effects on skeletal development and maintenance in men and women.

The advancement of androgen therapy has been limited by the inability to separate desirable androgenic activities from undesirable or dose limiting side effects. Recent advances in the development of selective estrogen receptor modulators (SERMs) which have a degree of tissue selectivity in targeting the estrogen receptor while eliminating or minimizing undesired side effects, suggests that a similar approach may be feasible for other NHR, such as, selective androgen receptor modulators (SARMs). See, for example, A. Negro-Vilar, J. Clin. Endocr. Metab 54(10):3459-3462 (1999); P. Reid et al., Investigational New Drugs 17:271-284 (1999).

To date, several approaches have been taken in order to unveil the androgen receptor function in vivo. The testicular feminized male (tfm) mouse (M. Lyon et al., Nature 227:1217-1219 (1970)), which represents an example of loss of function, possesses a single point mutation in the N-terminal region of the AR gene that results in a premature stop codon (M. Gaspar et al., Proc. Natl. Acad. Sci. U.S.A., 88:8606-8610 (1991)). Tfm mice are equivalent to complete androgen insensitive syndrome (cAIS) in man and are genetically considered to be males that are infertile. They are therefore unable to be used for the generation of mice genetically considered to be female homozygous for the AR gene mutation. By breeding tfm carrier females with males that were chimeric for the AR gene mutation, only a few number of homozygous tfm females were generated. Studies performed on these latter animals revealed that having a functional AR is not critical for their reproductive capabilities (M. Lyon et al., Proc. R. Soc. Lond. B. Biol. Sci., 208:1-12 (1980)). However, there is a limitation in the use of these animals for the elucidation of the AR function in the adult mouse. Since the animals lack the receptor throughout life, some of the observed phenotypes could be the result of the lack a functional AR during development. Conversely, a transgenic mouse line has been generated expressing the chloramphenicol acetyltransferase (CAT) reporter gene under the regulation of probasin, a prostate-specific promoter (Y. Yan et al., Prostate 32:129-139 (1997)). This and related lines are useful tools for the assessment of AR function, but they are limited to defining AR function in the prostate.

Therefore, it would be of interest to develop a transgenic non-human mammalian model for the assessment of tissue specific activity of the androgen receptor. Such a model could be used to study the tissue selective activity of pharmacological agents as well as the activity of the androgen receptor in different organs of males and females. The described invention herein represents such a model using a reporter gene under the control of an androgen-regulated promoter.

### SUMMARY OF THE INVENTION

The present invention provides a transgenic non-human mammal whose genome comprises a nucleic acid construct, wherein said construct comprises a reporter nucleic acid encoding a reporter operably linked to a promoter comprising an androgen response element (ARE), and said construct further comprises an androgen receptor nucleic acid encoding an androgen receptor, and wherein expression of said reporter nucleic acid is regulated by expression of said androgen receptor nucleic acid. In one aspect, the reporter is luciferase. In another aspect, the androgen response element is 2×DR-1.

The invention also provides a cell isolated from the transgenic mouse of the invention, wherein the genome of said cell comprises said nucleic acid construct.

The invention also provides a mouse cell line comprising the cell isolated from the transgenic mouse of the invention.

The invention also provides an isolated nucleic acid construct that comprises a reporter nucleic acid encoding a reporter operably linked to a promoter comprising an androgen response element (ARE), and said construct further comprises an androgen receptor nucleic acid encoding an androgen receptor, and wherein expression of said reporter nucleic acid is regulated by expression of said androgen receptor nucleic acid. In one aspect, the reporter is luciferase. In another aspect, the androgen response element is 2×DR-1.

The invention also includes a method for obtaining a target mouse whose genome comprises a nucleic acid construct, wherein said construct comprises a reporter nucleic acid encoding a reporter operably linked to a promoter comprising an androgen response element, and said construct further comprises an androgen receptor nucleic acid encoding an androgen receptor, and wherein expression of said reporter nucleic acid is regulated by expression of said androgen receptor nucleic acid, wherein said mouse can be bred to produce progeny mice whose genomes comprise said nucleic acid construct, said method comprising the steps of:

(a) solating a fertilized egg from a first female mouse;

(b) transferring a transgene comprising said nucleic acid construct into the fertilized egg;

(c) transferring the fertilized egg of step (b) to the uterus of a pseudopregnant second female mouse; and

(d) maintaining said second female mouse such that:

(i) said second female mouse becomes pregnant with an embryo derived from said fertilized egg of step (c);

(ii) said embryo develops into said target mouse; and

(iii) said target mouse is viably born from said second female mouse; wherein the genome of said target mouse comprises said nucleic acid construct and wherein said mouse can be bred to produce progeny mice whose genomes comprise said nucleic acid construct.

The invention also includes a method for producing a transgenic mouse cell line that expresses a reporter nucleic acid, said method comprising: (a) isolating cells from the transgenic mouse of the invention; and (b) placing the isolated cells under conditions to maintain growth and viability of the isolated cells such that said transgenic mouse cell line expresses said reporter nucleic acid.

The invention also provides a method of screening for a modulator of the androgen receptor, comprising administering a test substance to a transgenic non-human mammal of the invention and assaying the effect of said test substance on the activity of the androgen receptor. Modulators of the androgen receptor are particularly useful for treating a disorder associated with defective AR function, such as a cancer. In one aspect, the invention provides a method of identifying a test substance which is an antagonist or agonist of an androgen receptor, said method comprising: (a) determining the expression of said reporter in a transgenic mouse of the invention; (b) administering said test substance to a transgenic mouse of the invention and determining the expression of said reporter following said administering; (c) comparing the expression of said reporter in said step (a) and said step (b); wherein an increase in the expression of said reporter in said step (b) identifies said test substance as an agonist of said androgen receptor and wherein a decrease in the expression of said reporter is said step (b) identifies said test substance as an antagonist of said androgen receptor.

The invention further provides a transgenic non-human mammal whose genome comprises a nucleic acid construct, wherein said construct comprises a reporter nucleic acid encoding a reporter operably linked to a promoter comprising an androgen response element, and said construct further comprises an androgen receptor nucleic acid encoding an androgen receptor, and wherein said non-human mammal expresses said reporter nucleic acid in organs when said androgen receptor nucleic acid is expressed. Thus, expression of said reporter nucleic acid is regulated by expression of said androgen receptor nucleic acid.

The invention comprises a transgenic non-human mammal whose germ cells and somatic cells express a reporter gene under the regulation of a promoter capable of expressing androgen receptor, i.e., under the regulation of androgen response elements, and wherein said non-human mammal expresses luciferase in organs where the androgen receptor is activated. Cells in which the androgen receptor was activated can be readily determined by the expression of the reporter gene, which can be measured by standard bioluminescence imaging techniques known to those skilled in the art. In a preferred embodiment of the invention the non-human mammal is a mouse and the transgene construct comprises a reporter gene, comprising luciferase cDNA (SEQ ID NO: 2) regulated by a promoter containing two copies of the androgen response element DR-1 (2×DR-1) (SEQ ID NO: 3) and rat androgen receptor cDNA (SEQ ID NO: 4) regulated by the CMV promoter (SEQ ID NO: 5). The invention further comprises non-human mammalian embryos carrying the androgen-regulated reporter gene capable of developing into viable transgenic animals whose progeny carry the androgen-regulated reporter gene after breeding forward by sexual reproduction. The invention further comprises DNA constructs comprising selected promoters plus the reporter gene and the rat AR cDNA or DNA segments cloned into plasmids for ultimate insertion into the genome of a mammal.

The transgenic non-human mammals of the invention are characterized by the emission of light in tissues that contain an active androgen receptor. In a preferred embodiment of the invention the transgenic non-human mammals are utilized as a model or surrogate for human AR function for the identification and optimization of molecules and compounds that modulate androgen receptor activity. Molecules and compounds so identified can be used in the prevention and treatment of disorders associated with defective AR function including, but not limited to prostate cancer and andropausia. Thus, the invention provides an in vivo system to monitor the activity of the androgen receptor in different organs and tissues.

A further object of the present invention is to provide methods for identifying selective androgen receptor modulators (SARMs) that can act as antagonists or agonists in different tissues containing the androgen receptor. In one embodiment, antagonist activity in hormone-dependent tumors is ascertained via screening for inhibition of growth, either in vitro or in vivo, in hormone-dependent tumor cell lines. In another embodiment, the activity of potential SARM is also assessed in normal, non-tumor cell lines. Alternatively, an animal model expressing a hormone-dependent reporter gene can be used to assess the activity of a potential SARM in different tissues in the animal. Thus, the invention also embodies non-human mammals and methods for the identification of selective modulators of the androgen receptor and pharmaceutical compositions comprising the selective modulators so identified.

### BRIEF DESCRIPTION OF THE DRAWINGS:

FIG. 1 shows a diagrammatic representation of the transgene ARELUC/CMV-rAR construct (SEQ ID NO: 1). The luciferase cDNA (SEQ ID NO: 2) was cloned into a vector downstream of a promoter containing two androgen regulated elements (ARE-DR-1) and the SV40 promoter (SEQ ID NO: 3) and was flanked with intron and polyA sequences for efficient message processing. The rat androgen receptor cDNA (SEQ ID NO: 4) was cloned into the same plasmid downstream of the CMV promoter (SEQ ID NO: 5).

FIGS. 2A-2D show the nucleotide sequence of the ARE-LUC/CMV-rAR transgenic construct (SEQ ID NO: 1).

FIG. 3 shows the nucleotide sequence of the luciferase cDNA (SEQ ID NO: 2) The numbering corresponds to the position in the ARE-LUC/CMV-rAR construct (SEQ ID NO: 1)

FIG. 4 shows the nucleotide sequence of the 2×DR-1 SV40 promoter (SEQ ID NO: 3). The numbering corresponds to the position in the ARE-LUC/CMV-rAR construct (SEQ ID NO: 1).

FIG. 5 shows the nucleotide sequence of the rat androgen receptor cDNA (SEQ ID NO: 4). The numbering corresponds to the position in the ARE-LUC/CMVrAR construct (SEQ ID NO: 1).

FIG. 6 shows the nucleotide sequence of the CMV promoter (SEQ ID NO: 5). The numbering corresponds to the position in the ARE-LUC/CMV-rAR construct (SEQ ID NO: 1)

FIG. 7 shows a representative Northern blot analysis of the lung, heart, liver, and testis tissues of a control and three progeny mice found to have passed the transgene (ARE-LUC/CMV-rAR). Each lane contains 20 μg of total RNA isolated from the respective tissues resolved on a 1% agarose gel in 17.5% formaldehyde.

FIG. 8A shows a representative set of line 26 mice, one control and two transgenic mice, 15 minutes after being subcutaneously injected with 150 mg/kg of luciferin anesthetized, and placed in the Xenogen imaging system. Luciferase light emission was detected with a cooled CCD IVIS™ camera and represents the organs containing an active androgen receptor that induced the expression of the enzyme. FIG. 8B shows the same animals one week after the control and one of the transgenic mice (left and middle mouse, respectively) were castrated.

FIG. 9 shows the image captured with the charge-coupled device (CDD) IVIS™ camera (Xenogen Corporation, Alameda, Calif.) of the testis isolated from the transgenic mouse described in the FIG. 8A and the respective control.

FIGS. 10A-D show representative light emission pictures of control non-transgenic and transgenic pairs of mice from line 26 twenty four hours after being treated with testosterone (2 mg/kg).

FIG. 11 shows the luciferase activity measured in the different organ extracts from transgenic or non-transgenic mice treated or not treated with testosterone. Equal amounts of protein were assayed for the different groups.

FIG. 12 shows the effect of an androgen receptor antagonist, bicalutamide, (Casodex®, Astra Zeneca, London, UK) on the testosterone induced luciferase activity in quadriceps, bone, prostate/ seminal vesicles and kidney. Luciferase activity (cps) was measured in duplicates in equivalent protein samples (100 μg) of the corresponding organ extracts. The results are the average of three animals per group.

### DETAILED DESCRIPTION OF THE INVENTION

The androgen receptor is a hormone regulated transcription factor that controls the expression of many genetic programs involved in normal physiological processes, i.e., male sexual differentiation, as well as in pathological conditions such as prostate cancer. Those activities of the androgen receptor are cell type specific and depend on a number of cofactors that coexist in each one of those cell types.

The invention relates to the production of transgenic non-human mammals containing within their genomes a reporter gene, such as a luciferase reporter gene, whose expression is regulated by an activated androgen receptor, as well as an engineered vector designed to express functional androgen receptor. Upon injection of luciferin, luciferase's substrate from fireflies, the animals emit light from the tissues where the enzyme luciferase is produced, indicating activity of either the engineered or endogenous androgen receptor.

In a preferred embodiment of the invention, the reporter gene is luciferase, but those skilled in the art would know how to select and use other reporter genes including, but not limited to, green fluorescent protein (GFP), beta-galactosidase, beta-lactamase, chloramphemicol acetyltransferase (CAT), dopamine 2 receptor (D2R), thymidine kinase (TK), alkaline phosphatase (AP) or a generic tag detectable by ELISA. In the preferred embodiment, luciferase is used in the Ivis™ Imaging System (Xeragon Corporation, Alameda, Calif.).

Provided herein is the establishment of transgenic lines that express luciferase cDNA under the control of a promoter containing two direct repeat-1 androgen response elements (2×DR-1 AREs) (SEQ ID NO: 3) and rat androgen receptor cDNA (SEQ ID NO: 4) under the control of the cytomegalovirus (CMV) promoter (SEQ ID NO: 5). DR-1 is an 11-base pair sequence (5′ GGAACGGAACA 3′) (SEQ ID NO: 6), consisting of two potential core binding sites oriented as an overlapping direct repeat. DR-1 was identified as a potent androgen response element (ARE) by the binding of a human AR DNA-binding domain fusion protein to DNA in a random sequence selection assay (Z. Zhou et al., J. Biol. Chem., 272:8227-8235 (1997)). The placement in tandem of two copies of DR-1 demonstrated a strong preference for AR binding and transactivation when compared with the glucocorticoid receptor (Id.). Lines of mice were generated that expressed the transgene in multiple organs including, lung, heart, liver, and testis; mice harboring the transgene did not develop any abnormality. The transgenic animals described herein can be utilized in the identification, development, and optimization of biological and chemical moieties that modulate the activity of the androgen receptor. Such moieties in turn can be used for the treatment of, but not limited to, prostate cancer, andropausia, and hormone replacement.

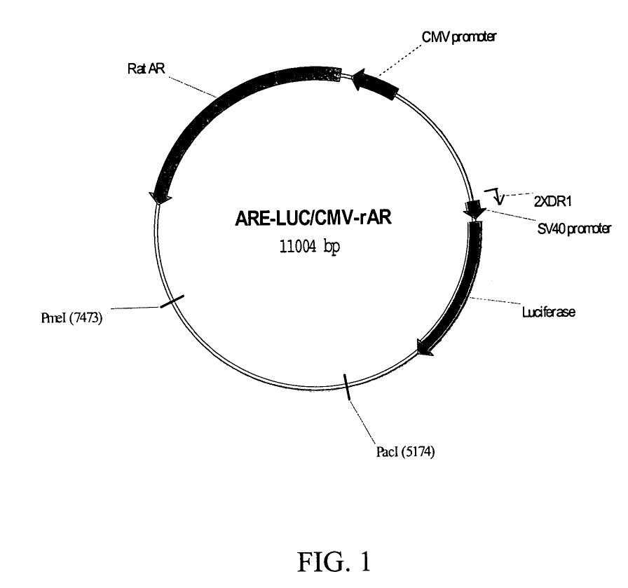

A construct was generated in which luciferase cDNA (SEQ ID NO: 2) from the pGL3 vector (Promega Corporation, Madison, Wis.) was placed under the regulation of a promoter containing two DR-1 AREs (SEQ ID NO: 3) (Z. Zhou et al., J. Biol. Chem., 272:8227-8235 (1997)) and was flanked with the chicken beta-globin intron and polyA sequences for efficient message processing. One skilled in the art would be able to select and use other AREs, in addition to DR-1, for use in regulatory luciferase expression. Separated by a stop transcription cassette, the same vector contains in an opposite orientation the CMV promoter (SEQ ID NO: 5) regulating the expression of the rat androgen receptor cDNA (SEQ ID NO: 4) as well as the SV40 virus intron and polyA sequences for efficient message processing (FIG. 1). The CMV promoter (SEQ ID NO: 5), when expressed in vivo in an animal, drives transcription of downstream sequences ubiquitously, in nearly every tissue. One skilled in the art would be able to clone the transgene of the invention into a vector under the control of other tissue specific promoters.

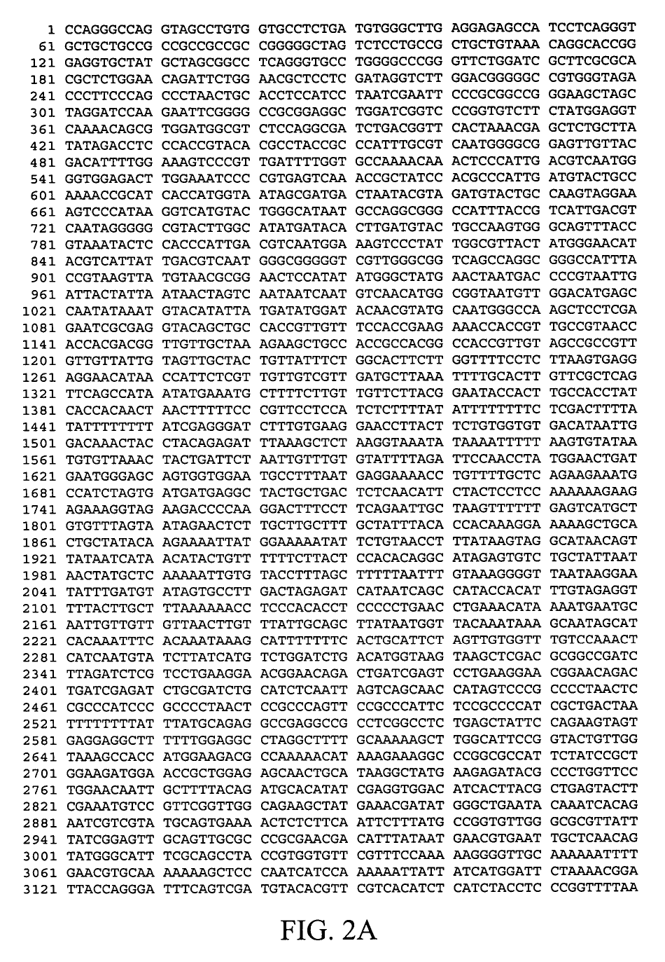

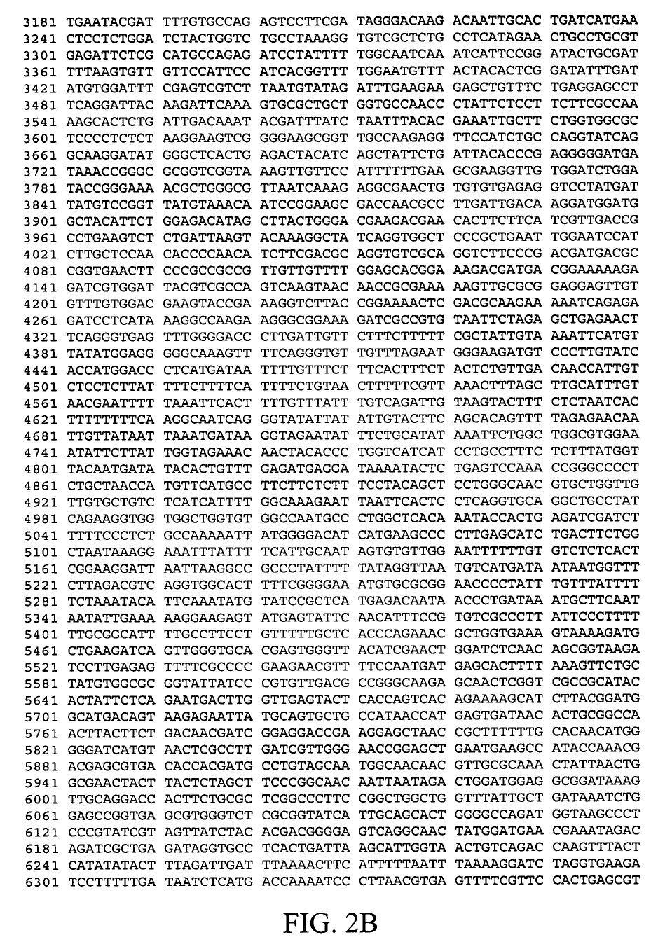

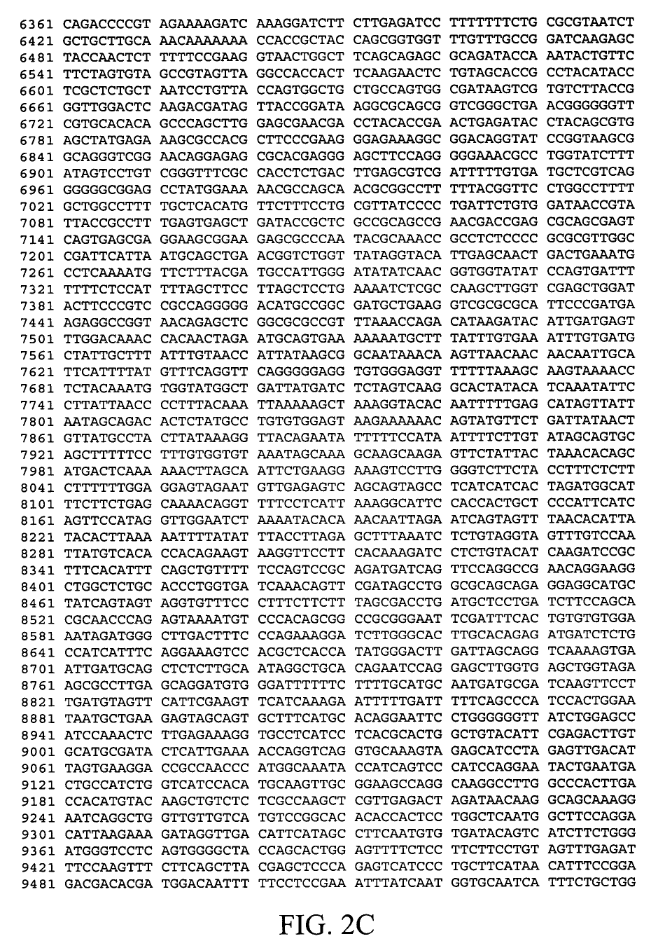

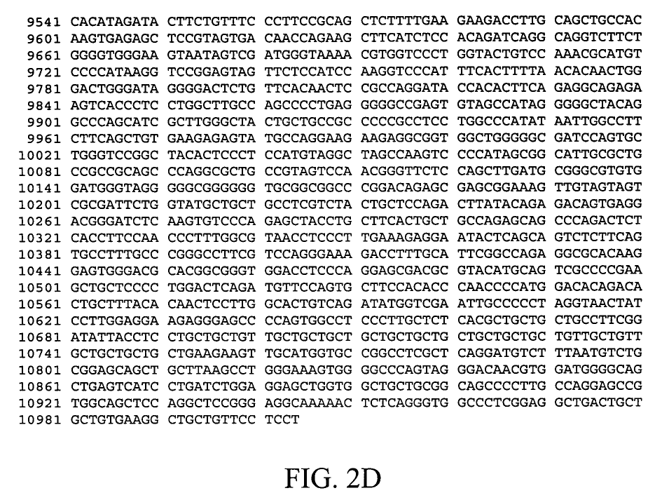

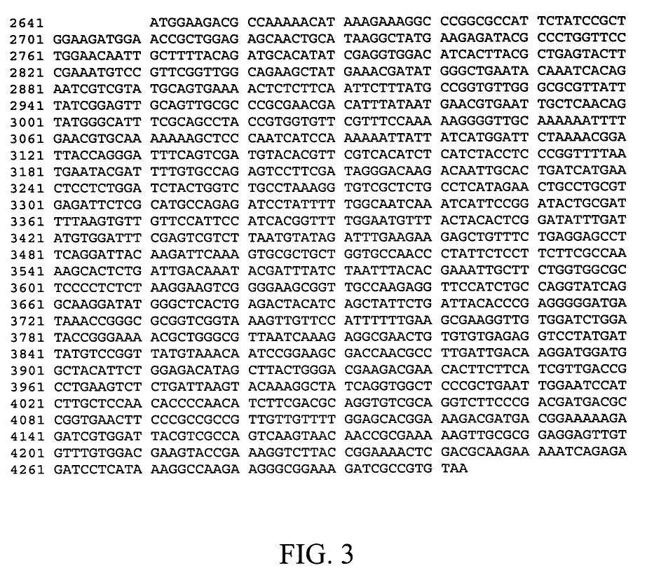

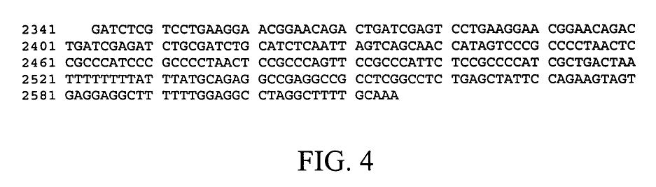

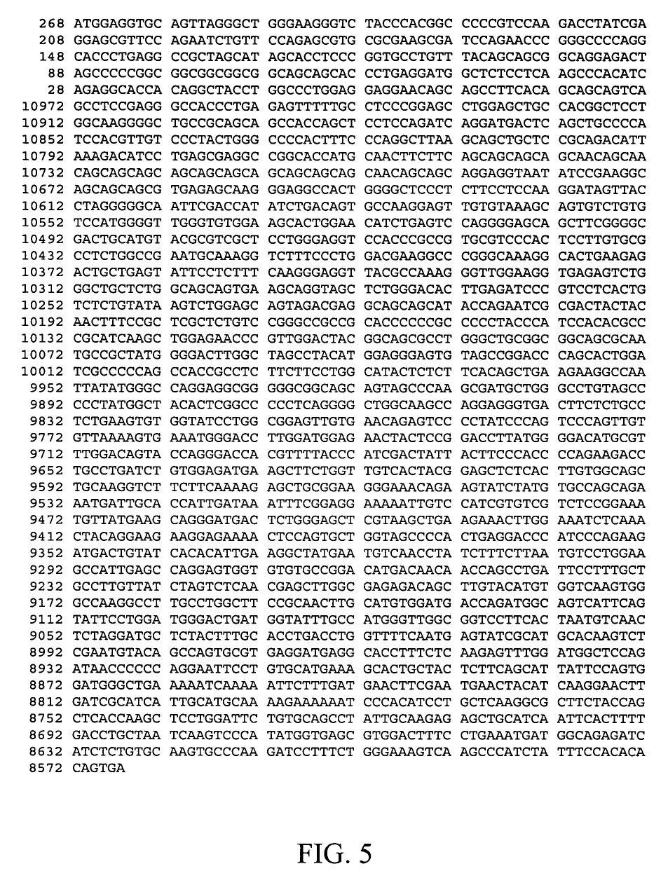

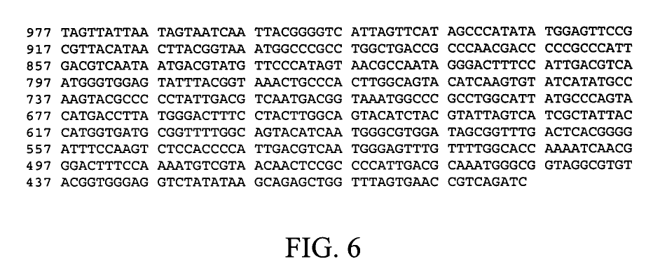

In a preferred embodiment described in the examples that follow the construct contained the engineered luciferase gene under the control of a promoter regulated by the androgen receptor (FIGS. 2A-2D, SEQ ID NO: 1). Those skilled in the art will recognize that other constructs can be generated that will be useful for the characterization of other members of the steroid nuclear hormone receptor family, such as the glucocorticoid, progesterone, mineralocorticoid, and estrogen receptors. By way of example, and not intending to be limited thereto, the transgene of the invention may comprise a promoter containing DR-1 androgen response elements, but other AREs, such as C3, PSA-AREs or probasin-AREs, or promoters containing glucocorticoid response elements, progesterone response elements, mineralocorticoid response elements or estradiol response elements.

The nucleotide sequences (cDNA) used herein were cloned using standard molecular biology techniques (Maniatus et al., Molecular Cloning: A Laboratory Manual, Cold Spring Harbor Laboratory (1982); Ausubel et al., Current Protocols in Molecular Biology, John Wiley and Sons, Volume 2 (1991)) based on sequences available in the public domain (e.g., GenBank).

The construct may also comprise selected nucleic acid regions associated with the transgene (as by fusion therewith) for mediation of, for example, its introduction into the target genome, its expression loci in the transgenic mammal, on/off external regulation of transgene expression, and other desired features, as generally known in the art.

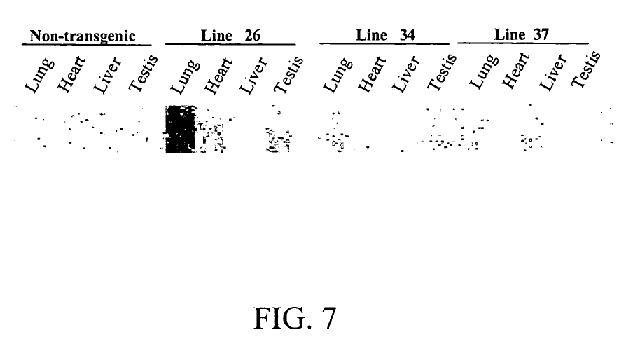

Microinjection of the above identified DNA construct into the pronucleus of fertilized oocytes resulted in the generation of four founder mice carrying the transgene (ARLuc) DNA. Of these four mice, three were found to pass the transgene in a Mendelian fashion to offspring. Mice from these three lines were subsequently examined for expression of the luciferase gene in multiple tissues. As can be seen in FIG. 7, one transgenic mouse line, identified as Line 26, had particularly high levels of the androgen responsive luciferase transgene expression in the lung, heart, and testis.

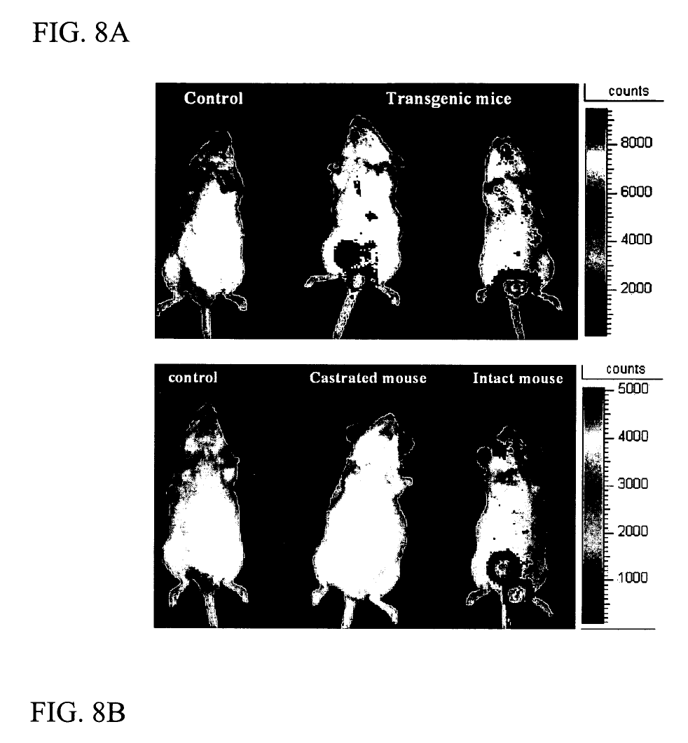

Three male transgenic mice from the line 26, one non-transgenic and two transgenic, were injected at 39 days old with 150 mg/kg body weight of luciferin 15 minutes prior to been anesthetized and placed in an IVIS™ Imaging System (Xenogen Corporate, Alameda, Calif.) where luciferase is detected with a cooled charge-coupled device (CCD) IVIS™ camera (Xenogen, Alameda, Calif.) and the images captured with Living Image® Software (Xenogen, Alameda, Calif.) designed by the manufacturer. As shown in FIGS. 8A and B, in contrast to the non-transgenic control, both transgenic mice present luminescence indicating luciferase expression predominantly in the genital area. In order to confirm that this expression was androgen dependent, the same study was repeated on the same animals one week after one of the transgenic mice was castrated. FIG. 8B shows the loss of luminescence in the castrated mouse, confirming the androgen dependent expression of the luciferase.

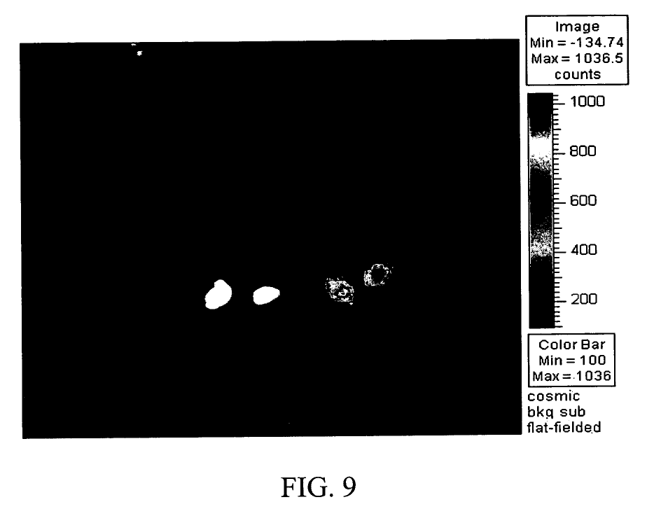

Without wishing to be bound by any theory, it was believed that the high luminescence observed in the genital area was due to the fact that the testis are the organs where androgens are synthesized and therefore the activity of the androgen receptor should be maximal. In order to test this belief, the testis from a control and transgenic mice were isolated after the animals were injected with 150 mg/kg luciferin, and exposed to the camera. As shown in FIG. 9, in contrast to the testes isolated from the non-transgenic animal, the testes from the transgenic animal emitted substantial light, which further confirms the androgen dependent regulation of the luciferase expression.

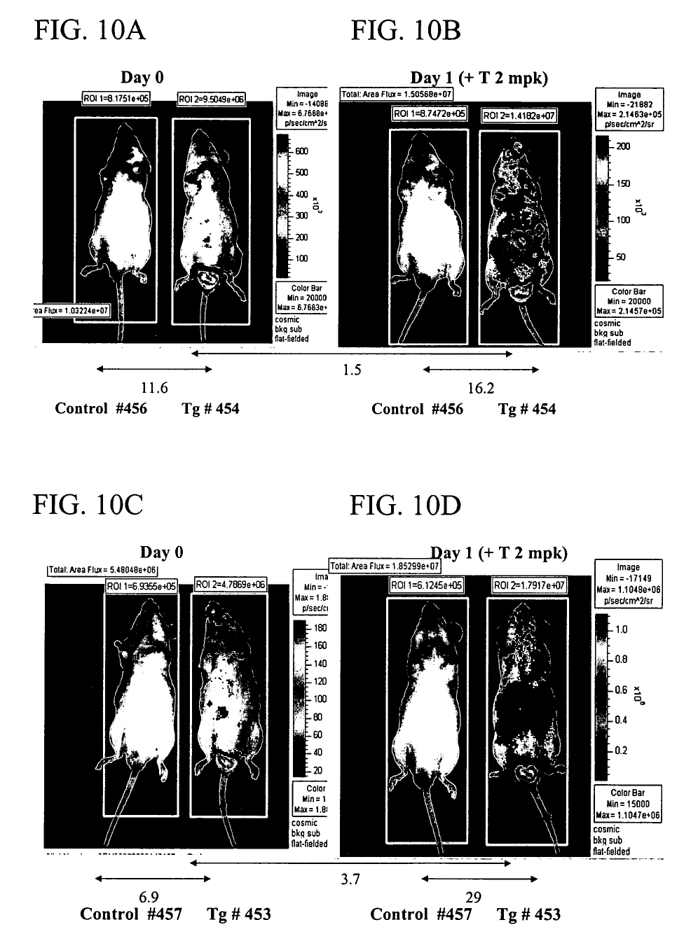

To further prove the effect of androgens on the expression of luciferase, two pairs of mice from line 26 were imaged at day 0 and at day 1 after receiving a subcutaneous injection of 2 mg/kg testosterone. As shown in FIGS. 10A-10D, testosterone treatment increased the total photon emission between 1.5 and 3.7 fold (comparing transgenic mice tag #454 and tag #453 with their baseline before treatment). The ratios for treated animals as compared to their non-transgenic controls were 16.2 and 29.0 folds, respectively.

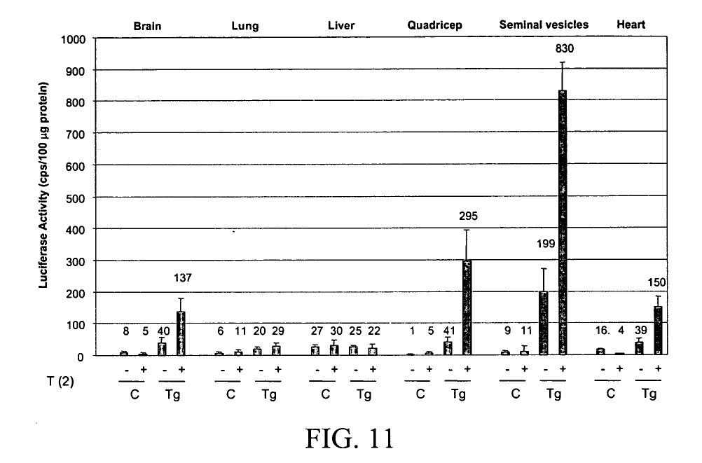

In order to test first, the possibility of measuring luciferase activity in single organs, and second, the ability to detect differences in the androgen function among them, luciferase activity was measured from total extracts prepared from brain, lung, liver, quadriceps, seminal vesicles, and heart. As shown in FIG. 11, testosterone treatment of transgenic mice promoted an increase in luciferase activity in brain (3.4 fold), quadriceps (7.2 fold), seminal vesicles (4.2 fold), and heart (3.8 fold) with respect to the extracts from their corresponding non-transgenic control.

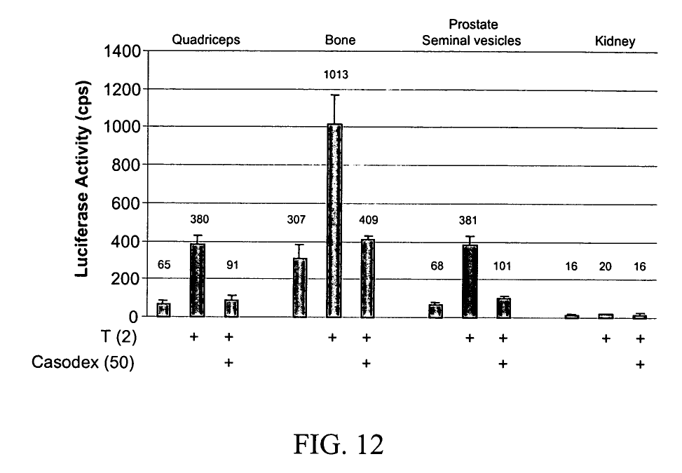

The transgenic animals of the invention are also useful for the development of compounds or pharmacotherapies for the treatment of disorders associated with defective androgen receptor function, particularly cancer. By “defective androgen receptor function” is meant any function resulting from aberrant expression, that is, either in an up-regulated or down-regulated manner, relevant to that of the wild type androgen receptor. To demonstrate this utility, three groups of mice were treated with testosterone (2 mg/kg, s.c.), testosterone and Casodex® (50 mg/kg, p.o.), or untreated, respectively. As shown in FIG. 12, bicalutamide (Casodex®) inhibited testosterone induced luciferase expression in quadriceps, bone, prostate-seminal vesicles with an acceptable dynamic range (92% inhibition of the testosterone effect (5.8 fold induction over untreated) in quadriceps, 86% inhibition of the testosterone effect (3.3 fold induction over untreated) in bone, and 90% inhibition of the testosterone effect (5.6 fold induction over untreated) in prostate-seminal vesicles). No response was observed in kidney.

Having now generally described the invention, the same will be more readily understood through reference to the following examples, which are provided by way of illustration and are not intended to be limiting of the present invention. EXAMPLES:

1. ARE-LUC/CMV-rAR Transgene Construct

A. pGL3/2×DR-1 Luciferase

Equimolar amounts of the complementary oligonucleotides DR-1(F) (ARE) (SEQ ID NO: 7) and DR-1(R) (ARE) (SEQ ID NO: 8) were annealed and then ligated into the XhoI digested pGL3-Promoter plasmid (Promega Corporation, Madison, Wis.). The oligonucleotide DR-1 (F) (ARE) (SEQ ID NO: 7) has the sequence: 5′TCGAGTCCTGAAGGAACGGAACAGACTGA-3′. The oligonucleotide DR-1(R) (ARE) has the sequence: 5′-TCGATCAGTCTGTTCCGTTCCTTCAGGAC-3′ (SEQ ID NO: 8). A second DR-1 response element was inserted upstream of the existing DR-1 element in pGL3/1×DR-1/luciferase by annealing equimolar amounts of the complementary oligonucleotide 1×DR-1(F) (SEQ ID NO: 9) and 1×DR-1(R) (SEQ ID NO: 10) and then ligating both into the SacI/XhoI digested pGL3/1×DR1/luciferase plasmid. The oligonucleotide 1×DR-1(F) (SEQ ID NO: 9) has the sequence:

5′-CGTCCTGAAGGAACGGAACAGACTGA-3′. The oligonucleotide 1×DR-1(R) (SEQ ID NO: 10) has the sequence: 5′-TCGATCAGTCTGTTCCGTTTTTCCTTCAGGACGAGCT-3′.

B. ARE-LUC/CMV-rAR

The generation of the expression construct was done using standard molecular biology techniques, for example, Ausubel et al., Current Protocols in Molecular Biology, John Wiley and Sons, Volume 2 (1991). The complete sequence of ARE-LUC/CMV-rAR transgene construct (SEQ NO: 1) is shown in the FIGS. 2A-2D.

A NotI fragment comprising the nucleotide sequence of SEQ ID NO: 4 and encoding the complete amino acid sequence of the rat androgen receptor was isolated from pcDNA-rAR and blunted using Klenow. The fragment was then cloned into Smal/Afel restricted pCMV-TSIR to create the intermediate pCMV-rARtemp. The plasmid pTetInd was restricted with NotI and BglII, blunted with Klenow, and ligated upon itself. The resulting plasmid was subsequently digested with EcoRV and XbaI and used as a vector for subcloning of the EcoICRI/XbaI fragment isolated following digestion of pGL3-pro/2×DR-1. This fragment comprised an androgen responsive promoter, which was generated by fusing two androgen response elements to the 5-prime end of a minimal SV40 promoter (SEQ ID NO: 3), as well as sequences encoding the full length luciferase protein (SEQ ID NO: 2). The resulting plasmid was designated p2×DR-1-Luctemp-1. A stop transcription cassette flanked by an XhoI site at the 5′ end and a SalI site at the 3′ end was generated by PCR using pBS302 as a template. The XhoI/SalI restricted PCR fragment was subcloned into XhoI restricted p2×DR-1-LucTemp 1 in the orientation such that the 3′ end of the stop cassette was inserted just upstream of the 5′ end of the androgen responsive promoter. The resulting plasmid was designated p2×DR-1-Luctemp-2. This plasmid was then digested with XhoI and XbaI and the fragment containing the stop transcription cassette, androgen responsive promoter, and the sequences encoding the luciferase protein was inserted into XhoI/XbaI restricted pCMV-ARtemp. This resulted in completion of the ARE-LUC/CMV-rAR plasmid. The 8.6 kb DNA fragment generated by PmeI/PacI digestion of ARE-LUC/CMV-rAR was isolated for microinjection into mouse embryos in order to create the ARLuc transgenic animals.

2. Generation and Breeding of Transgenic Mice

Transgenic mice harboring the ARE-LUC/CMV-rAR construct were generated by microinjection of a PmeI/PacI fragment from the above construct into the pronucleus of C57B1/6×DBA2 F2 (B6D2F2) embryos. Embryos were generated by in-house mating of hybrid stud B6D2 males to virgin females from the same background (Harlan Sprague Dawley, Indianapolis, Ind.) using the techniques described by Hogan et al., Manipulating the Mouse Embryo: a Laboratory Manual, second edition, Brigid Hogan, Rosa Beddington, Frank Constantini, and Elizabeth Lacey, eds, Cold Spring Harbor Laboratory Press (1994). Injected embryos were transferred to pseudopregnant ICR female mice (Harlan Sprague Dawley, Indianapolis, Ind.) and allowed to develop to term. At five to eight days of age toe and tail samples were taken for DNA analysis of the transgene. Mice harboring the transgene were identified by a polyrnerase chain reaction (PCR) strategy designed to detect the insulator stop cassette sequences intervening between the CMV promoter and the DR-I sequences in the vector, upstream primer 5′ CTTGGCTTGCTTTGCTATTTA3′ (SEQ NO: 11) and downstream primer 5′ATGTGGTATGGCTGATTATGA3′ (SEQ NO: 12). Founder mice (F0) shown to harbor the transgene were then outbred to the ICR background, and progeny (F1) were again tested for transmission of the transgene in a Mendelian fashion. All mice were housed in shoebox housing with food and water ad lib on a 12/12 light dark cycle, and were humanely handled under the guidelines of the institutional ACUC in an AAALAC accredited facility.

3. Gene Expression Analysis

Detection of ARE-LUC/CMV-rAR transgene expression was performed by Northern blot analysis. To identify transgenic lines that had expression of the transgene, F1 offspring from founders were euthanized and their lung, heart, liver, and testis were harvested. The samples were flash frozen in liquid nitrogen and stored until the time of mRNA isolation. Total RNA was isolated using a monophasic solution of phenol and guanidine isothiocyanates, such as the Trizol® LS Reagent system as described by the manufacturer (GIBCO™ Invitrogen Corporation, Carlsbad, Calif.). From these RNA isolates, MRNA was extracted using the Oligotex Direct mRNA kit as recommended by the manufacturer (Operon/QIAGEN, Valencia, Calif.). The mRNA was resolved in 1.0% agarose gel electrophoresis under denaturing conditions with 17.6% formaldehyde and transferred to nylon membrane (Hybond-N, Amersham Biosciences, Uppsala, Sweden) by capillary blotting before hybridization. ARE-LUC/CMV-rAR messages were detected by hybridizing the RNA to a 607 bp radiolabelled probe designed to detect the luciferase mRNA (Ready-to-Go kit, New England Nuclear/Perkin Elmer™ Life Sciences, Boston, Mass.). The 607 bp fragment was generated by PCR using the pGL3 vector as template and the oligonucleotides LUC (F): 5′-GGTAACCCAGTAGATCCAGAG-3′ (SEQ ID NO: 13) and LUC (R): 5′-GGAAGACGCCAAAAACATAAAG-3′ (SEQ ID NO: 14). Hybridization was done in Rapid-hyb buffer (Amersham Biosciences, Uppsala, Sweden) overnight and nonspecific annealing of the probe was eliminated by multiple washes under stringent conditions (2×20 min in 0.1×SSC, 2%SDS at 65° C.). Specific hybridization of the probe to the luciferase message was detected on a phosphoimager (Model FLA-2000, Mfr. Fuji Film, Stanford, Conn.).

4. Luciferase Imaging in Vivo

Mice designated for detection of luciferase expression by in vivo imaging are injected with 150 mg/kg luciferin in PBS 15 minutes prior to imaging. Subsequently, the mice are placed under chemical restraint by injection with avertin (0.3 ml of a 2.5% solution in PBS). Anesthetized mice are placed in the IVIS™ Imaging System (Xenogen Corporation, Alameda, Calif.), a dark box containing a cooled CCD IVIS™ camera and stage. After image acquisition of two minutes, the images are processed with Living Image® Software (Xenogen, Alameda, Calif.). For imaging of tissues, the mice are injected with luciferin 15 minutes prior to euthanasia via carbon dioxide, and the tissues excised and imaged accordingly.

5. Measurement of Luciferase Activity in Total Organ Extracts.

Heart, lung, liver, muscle, brain, bone, seminal vesicles (including prostate), and testis were collected and flash frozen until assay time. Extracts were prepared and luciferase activity was assessed using the luciferase assay kit (LUC1-1KT) (Sigma). Tissues were ground to a fine powder by mortar and pestle in the presence of liquid nitrogen and then placed in 1 ml of lysis buffer. Insoluble material was spun out at 14,000 rpm for ten minutes. (Eppendorf microfuge). The top layer of fat was discarded and the supernatant was collected. A Bradford assay (M. Bradford, Anal. Biochem. 1976, 72: 248-254) was done in triplicate to determine protein concentration. One hundred μg of total protein per tissue was added to 100 μl of assay buffer containing the luciferase substrate in triplicate. Chemiluminesence was measured in costar black 96 well plates by a Beckman top count in counts per second.

1. A transgenic non-human mammal whose genome comprises a nucleic acid construct, wherein said construct comprises a reporter nucleic acid encoding a reporter operably linked to a promoter comprising an androgen response element (ARE), and said construct further comprises an androgen receptor nucleic acid encoding an androgen receptor, and wherein expression of said reporter nucleic acid is regulated by expression of said androgen receptor nucleic acid.

2. The transgenic non-human mammal of claim 1 wherein said reporter is luciferase.

3. The transgenic non-human mammal of claim 1 wherein said androgen response element is 2×DR-1.

4. A cell isolated from the transgenic mouse of claim 1, wherein the genome of said cell comprises said nucleic acid construct.

5. The cell of claim 4 wherein said reporter is luciferase.

6. The cell of claim 4 wherein said androgen response element is 2×DR-1.

7. A mouse cell line comprising the cell of claim 4.

8. An isolated nucleic acid construct that comprises a reporter nucleic acid encoding a reporter operably linked to a promoter comprising an androgen response element (ARE), and said construct further comprises an androgen receptor nucleic acid encoding an androgen receptor, and wherein expression of said reporter nucleic acid is regulated by expression of said androgen receptor nucleic acid.

9. The construct of claim 8 wherein said reporter is luciferase.

10. The construct of claim 8 wherein said androgen response element is 2×DR-1.

11. A method for obtaining a target mouse whose genome comprises a nucleic acid construct, wherein said construct comprises a reporter nucleic acid encoding a reporter operably linked to a promoter comprising an androgen response element (ARE), and said construct further comprises an androgen receptor nucleic acid encoding an androgen receptor, and wherein expression of said reporter nucleic acid is regulated by expression of said androgen receptor nucleic acid,
wherein said mouse can be bred to produce progeny mice whose genomes comprise said nucleic acid construct, said method comprising the steps of:
(a) solating a fertilized egg from a first female mouse;
(b) transferring a transgene comprising said nucleic acid construct into the fertilized egg;
(c) transferring the fertilized egg of step (b) to the uterus of a pseudopregnant second female mouse; and
(d) maintaining said second female mouse such that:
(i) said second female mouse becomes pregnant with an embryo derived from said fertilized egg of step (c);
(ii) said embryo develops into said target mouse; and
(iii) said target mouse is viably born from said second female mouse;
wherein the genome of said target mouse comprises said nucleic acid construct and wherein said mouse can be bred to produce progeny mice whose genomes comprise said nucleic acid construct.

12. A method for producing a transgenic mouse cell line that expresses a reporter nucleic acid, said method comprising:
(a) isolating cells from the transgenic mouse of claim 1; and
(b) placing the isolated cells under conditions to maintain growth and viability of the isolated cells such that said transgenic mouse cell line expresses said reporter nucleic acid.

13. A method of screening for a modulator of the androgen receptor, comprising administering a test substance to the transgenic non-human mammal of claim 1 and assaying the effect of said test substance on the activity of the androgen receptor.

14. A transgenic non-human mammal whose genome comprises a nucleic acid construct, wherein said construct comprises a reporter nucleic acid encoding a reporter operably linked to a promoter comprising an androgen response element (ARE), and said construct further comprises an androgen receptor nucleic acid encoding an androgen receptor, and wherein said non-human mammal expresses said reporter nucleic acid in organs when said androgen receptor nucleic acid is expressed.


SEQUENCE LISTING (14 sequences)

SEQ ID NO: 1 - DNA, 11004 bp, Artificial
  artificial nucleic acid construct
ccagggccaggtagcctgtggtgcctctgatgtgggcttgaggagagccatcctcagggtgctgctgccgccgccgccgccgggggctagtctcctgccgctgctgtaaacaggcaccgggaggtgctatgctagcggcctcagggtgcctggggcccgggttctggatcgcttcgcgcacgctctggaacagattctggaacgctcctcgataggtcttggacgggggccgtgggtagacccttcccagccctaactgcacctccatcctaatcgaattcccgcggccgggaagctagctaggatccaagaattcggggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtaatgttggacatgagccaatataaatgtacatattatgatatggatacaacgtatgcaatgggccaagctcctcgagaatcgcgaggtacagctgccaccgttgtttccaccgaagaaaccaccgttgccgtaaccaccacgacggttgttgctaaagaagctgccaccgccacggccaccgttgtagccgccgttgttgttattgtagttgctactgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgacttttatattttttttatcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagctcgacgcggccgatcttagatctcgtcctgaaggaacggaacagactgatcgagtcctgaaggaacggaacagactgatcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattaattcactcctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggattaattaaggccgccctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgaacggtctggttataggtacattgagcaactgactgaaatgcctcaaaatgttctttacgatgccattgggatatatcaacggtggtatatccagtgatttttttctccattttagcttccttagctcctgaaaatctcgccaagcttggtcgagctggatacttcccgtccgccagggggacatgccggcgatgctgaaggtcgcgcgcattcccgatgaagaggccggtaacagagctcggcgcgccgtttaaaccagacataagatacattgatgagtttggacaaaccacaactagaatgcagtgaaaaaaatgctttatttgtgaaatttgtgatgctattgctttatttgtaaccattataagcggcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttctgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatcctctgtacatcaagatccgctttcacatttcagctgtttttccagtccgcagatgatcagttccaggccgaacaggaaggctggctctgcaccctggtgatcaaacagttcgatagcctggcgcagcagaggaggcatgctatcagtagtaggtgtttccctttcttctttagcgacctgatgctcctgatcttccagcacgcaacccagagtaaaatgtcccacagcggccgcgggaattcgatttcactgtgtgtggaaatagatgggcttgactttcccagaaaggatcttgggcacttgcacagagatgatctctgccatcatttcaggaaagtccacgctcaccatatgggacttgattagcaggtcaaaagtgaattgatgcagctctcttgcaataggctgcacagaatccaggagcttggtgagctggtagaagcgccttgagcaggatgtgggattttttcttttgcatgcaatgatgcgatcaagttccttgatgtagttcattcgaagttcatcaaagaatttttgatttttcagcccatccactggaataatgctgaagagtagcagtgctttcatgcacaggaattcctggggggttatctggagccatccaaactcttgagaaaggtgcctcatcctcacgcactggctgtacattcgagacttgtgcatgcgatactcattgaaaaccaggtcaggtgcaaagtagagcatcctagagttgacattagtgaaggaccgccaacccatggcaaataccatcagtcccatccaggaatactgaatgactgccatctggtcatccacatgcaagttgcggaagccaggcaaggccttggcccacttgaccacatgtacaagctgtctctcgccaagctcgttgagactagataacaaggcagcaaaggaatcaggctggttgttgtcatgtccggcacacaccactcctggctcaatggcttccaggacattaagaaagataggttgacattcatagccttcaatgtgtgatacagtcatcttctgggatgggtcctcagtggggctaccagcactggagttttctccttcttcctgtagtttgagatttccaagtttcttcagcttacgagctcccagagtcatccctgcttcataacatttccggagacgacacgatggacaatttttcctccgaaatttatcaatggtgcaatcatttctgctggcacatagatacttctgtttcccttccgcagctcttttgaagaagaccttgcagctgccacaagtgagagctccgtagtgacaaccagaagcttcatctccacagatcaggcaggtcttctggggtgggaagtaatagtcgatgggtaaaacgtggtccctggtactgtccaaacgcatgtccccataaggtccggagtagttctccatccaaggtcccatttcacttttaacacaactgggactgggataggggactctgttcacaactccgccaggataccacacttcagaggcagagaagtcaccctcctggcttgccagcccctgagggggccgagtgtagccatagggggctacaggcccagcatcgcttgggctactgctgccgcccccgcctcctggcccatataattggccttcttcagctgtgaagagagtatgccaggaagaagaggcggtggctgggggcgatccagtgctgggtccggctacactccctccatgtaggctagccaagtccccatagcggcattgcgctgccgccgcagcccaggcgctgccgtagtccaacgggttctccagcttgatgcgggcgtgtggatgggtagggggcggggggtgcggcggcccggacagagcgagcggaaagttgtagtagtcgcgattctggtatgctgctgcctcgtctactgctccagacttatacagagacagtgaggacgggatctcaagtgtcccagagctacctgcttcactgctgccagagcagcccagactctcaccttccaaccctttggcgtaacctcccttgaaagaggaatactcagcagtctcttcagtgcctttgcccgggccttcgtccagggaaagacctttgcattcggccagaggcgcacaaggagtgggacgcacggcgggtggacctcccaggagcgacgcgtacatgcagtcgccccgaagctgctcccctggactcagatgttccagtgcttccacacccaaccccatggacacagacactgctttacacaactccttggcactgtcagatatggtcgaattgccccctaggtaactatccttggaggaagagggagccccagtggcctcccttgctctcacgctgctgctgccttcggatattacctcctgctgctgttgctgctgctgctgctgctgctgctgctgctgttgctgttgctgctgctgctgaagaagttgcatggtgccggcctcgctcaggatgtctttaatgtctgcggagcagctgcttaagcctgggaaagtggggcccagtagggacaacgtggatggggcagctgagtcatcctgatctggaggagctggtggctgctgcggcagccccttgccaggagccgtggcagctccaggctccgggaggcaaaaactctcagggtggccctcggaggctgactgctgctgtgaaggctgctgttcctcct

SEQ ID NO: 2 - DNA, 1653 bp, Artificial
  luciferase
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 3 - DNA, 272 bp, Artificial
  2XDR-1 and SV40 promoter
gatctcgtcctgaaggaacggaacagactgatcgagtcctgaaggaacggaacagactgatcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 4 - DNA, 2706 bp, Rattus norvegicus
atggaggtgcagttagggctgggaagggtctacccacggcccccgtccaagacctatcgaggagcgttccagaatctgttccagagcgtgcgcgaagcgatccagaacccgggccccaggcaccctgaggccgctagcatagcacctcccggtgcctgtttacagcagcggcaggagactagcccccggcggcggcggcggcagcagcaccctgaggatggctctcctcaagcccacatcagaggcaccacaggctacctggccctggaggaggaacagcagccttcacagcagcagtcagcctccgagggccaccctgagagtttttgcctcccggagcctggagctgccacggctcctggcaaggggctgccgcagcagccaccagctcctccagatcaggatgactcagctgccccatccacgttgtccctactgggccccactttcccaggcttaagcagctgctccgcagacattaaagacatcctgagcgaggccggcaccatgcaacttcttcagcagcagcagcaacagcaacagcagcagcagcagcagcagcagcagcagcaacagcagcaggaggtaatatccgaaggcagcagcagcgtgagagcaagggaggccactggggctccctcttcctccaaggatagttacctagggggcaattcgaccatatctgacagtgccaaggagttgtgtaaagcagtgtctgtgtccatggggttgggtgtggaagcactggaacatctgagtccaggggagcagcttcggggcgactgcatgtacgcgtcgctcctgggaggtccacccgccgtgcgtcccactccttgtgcgcctctggccgaatgcaaaggtctttccctggacgaaggcccgggcaaaggcactgaagagactgctgagtattcctctttcaagggaggttacgccaaagggttggaaggtgagagtctgggctgctctggcagcagtgaagcaggtagctctgggacacttgagatcccgtcctcactgtctctgtataagtctggagcagtagacgaggcagcagcataccagaatcgcgactactacaactttccgctcgctctgtccgggccgccgcaccccccgccccctacccatccacacgcccgcatcaagctggagaacccgttggactacggcagcgcctgggctgcggcggcagcgcaatgccgctatggggacttggctagcctacatggagggagtgtagccggacccagcactggatcgcccccagccaccgcctcttcttcctggcatactctcttcacagctgaagaaggccaattatatgggccaggaggcgggggcggcagcagtagcccaagcgatgctgggcctgtagccccctatggctacactcggccccctcaggggctggcaagccaggagggtgacttctctgcctctgaagtgtggtatcctggcggagttgtgaacagagtcccctatcccagtcccagttgtgttaaaagtgaaatgggaccttggatggagaactactccggaccttatggggacatgcgtttggacagtaccagggaccacgttttacccatcgactattacttcccaccccagaagacctgcctgatctgtggagatgaagcttctggttgtcactacggagctctcacttgtggcagctgcaaggtcttcttcaaaagagctgcggaagggaaacagaagtatctatgtgccagcagaaatgattgcaccattgataaatttcggaggaaaaattgtccatcgtgtcgtctccggaaatgttatgaagcagggatgactctgggagctcgtaagctgaagaaacttggaaatctcaaactacaggaagaaggagaaaactccagtgctggtagccccactgaggacccatcccagaagatgactgtatcacacattgaaggctatgaatgtcaacctatctttcttaatgtcctggaagccattgagccaggagtggtgtgtgccggacatgacaacaaccagcctgattcctttgctgccttgttatctagtctcaacgagcttggcgagagacagcttgtacatgtggtcaagtgggccaaggccttgcctggcttccgcaacttgcatgtggatgaccagatggcagtcattcagtattcctggatgggactgatggtatttgccatgggttggcggtccttcactaatgtcaactctaggatgctctactttgcacctgacctggttttcaatgagtatcgcatgcacaagtctcgaatgtacagccagtgcgtgaggatgaggcacctttctcaagagtttggatggctccagataaccccccaggaattcctgtgcatgaaagcactgctactcttcagcattattccagtggatgggctgaaaaatcaaaaattctttgatgaacttcgaatgaactacatcaaggaacttgatcgcatcattgcatgcaaaagaaaaaatcccacatcctgctcaaggcgcttctaccagctcaccaagctcctggattctgtgcagcctattgcaagagagctgcatcaattcacttttgacctgctaatcaagtcccatatggtgagcgtggactttcctgaaatgatggcagagatcatctctgtgcaagtgcccaagatcctttctgggaaagtcaagcccatctatttccacacacagtga

SEQ ID NO: 5 - DNA, 589 bp, Artificial
  CMV promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 6 - DNA, 11 bp, Artificial
  DR-1
ggaacggaaca

SEQ ID NO: 7 - DNA, 29 bp, Artificial
  DR-1(F)
tcgagtcctgaaggaacggaacagactga

SEQ ID NO: 8 - DNA, 29 bp, Artificial
  DR-1(R)
tcgatcagtctgttccgttccttcaggac

SEQ ID NO: 9 - DNA, 26 bp, Artificial
  1XDR-1(F)
cgtcctgaaggaacggaacagactga

SEQ ID NO: 10 - DNA, 37 bp, Artificial
  1XDR-1(R)
tcgatcagtctgttccgtttttccttcaggacgagct

SEQ ID NO: 11 - DNA, 21 bp, Artificial
  upstream primer
cttggcttgctttgctattta

SEQ ID NO: 12 - DNA, 21 bp, Artificial
  downstream primer
atgtggtatggctgattatga

SEQ ID NO: 13 - DNA, 21 bp, Artificial
  LUC (F)
ggtaacccagtagatccagag

SEQ ID NO: 14 - DNA, 22 bp, Artificial
  LUC (R)
ggaagacgccaaaaacataaag